(19) United States
(12) Patent Application Publication
(10) Pub. No.: US 2022/0208377 A1
(43) Pub. Date: Jun. 30, 2022

(54) Patient Post-Operation Improvement Prediction Using Machine Learning

(71) Applicant: Universal Research Solutions, LLC, Columbia, MO (US)

(72) Inventor: Otho Raymond Plummer, Columbia, MO (US)

(21) Appl. No.: 17/566,289

(22) Filed: Dec. 30, 2021

Related U.S. Application Data
(60) Provisional application No. 63/132,452, filed on Dec. 30, 2020.

(51) Int. Cl. G16H 50/20; G16H 30/40

(52) U.S. Cl. CPC G16H 50/20; G16H 30/40

(57) ABSTRACT

Systems, methods, and computer programs for predicting patient post-operation improvement are disclosed. In one aspect, a method includes generating input data that comprises first data indicative of a perception of a patient and second data indicative of a morphological variable of the patient, providing the input data to a machine learning model that has been trained to generate output data indicative of a patient's post-operation improvement based on processing of data describing a perception of a patient and second a morphological variable of the patient, processing the provided input data through the machine learning model to generate output data indicative of patient's post-operation improvements, determining, based on the generated output data, a level of post-operation improvement for the patient, and generating, recommendation data for the patient indicating whether the patient is to undergo an operation, based on the determined level of post-operation improvement.

### CROSS-REFERENCE TO RELATED APPLICATION

This application claims the benefit of U.S. Provisional Patent Application No. 63/132,452 filed Dec. 30, 2020, the entirety of which is incorporated herein by reference.

### BACKGROUND

Many medical procedures come with an associated risk. Determining an accurate likelihood of success of a procedure is of considerable interest.

### SUMMARY

According to one innovative aspect of the present disclosure, a method for predicting the post-operation improvement of a patient is disclosed. The method can include actions of obtaining, by one or more computers, a first set of data indicative of one or more perceptions of a patient, obtaining, by one or more computers, a second set of data indicative of one or more morphological variables of the patient, generating, by one or more computers, input data that comprises the first data set and the second data set, providing, by one or more computers, the generated input data to a machine learning model that has been trained to generate output data indicative of a patient's post-operation improvement based on processing of input data that includes (i) a first data set indicative of one or more perceptions of a patient and (ii) second data indicative of one or more morphological variables of the patient, processing, by one or more computers, the provided input data through the machine learning model in order to generate output data that includes a set of one or more scores indicative of patient's post-operation improvements based on the provided first set of data and the second set of data, determining, by one or more computers and based on the generated output data, a level of post-operation improvement for the patient, and generating, by one or more computers, data corresponding to a recommendation for the patient whether to undergo the operation or not, based on the determined level of poser operation improvement for the patient.

Other versions include corresponding systems, apparatus, and computer programs to perform the actions of methods defined by instructions encoded on computer storage devices.

These and other versions may optionally include one or more of the following features. For instance, in some implementations, the first set of data can include a numerical representations one or more of a patient age at time of surgery, sex, operative side, preoperative VAS pain score, preoperative ASES responses to the ten activity-specific ASES function questions, or an ASES total score.

In some implementations, the second set of data includes a 2D CT scan that indicate one or more of Walch type, Goutallier classification, or a tangent sign.

In some implementations, the second set of data can include a 3D CT reconstruction that indicates one or more of a Walch type, a Goutallier classification, or a tangent sign.

In some implementations, the second set of data includes a numerical representation of one or more of a Walch type, a Goutallier classification, or a tangent sign.

In some implementations, the machine learning model is one or more of a support vector machine (SVM), a random forest, a logistic regression classifier, a hidden Markov model, a linear regression model.

In some implementations, the machine learning model is a convolutional neural network. In such implementations, processing, by one or more computers, the provided input data through each layer of the machine learning model can include processing, by one or more computers, the provided input data through each layer of the machine learning model the generated input data through each layer of the convolutional neural network.

These and other innovative aspects of the present disclosure are described in more detail in the accompanying drawings, the detailed description below, and in the accompanying claims.

### BRIEF DESCRIPTION OF THE DRAWINGS

FIG. 1 is an example an example prediction system for predicting patient post-operation improvement.

FIG. 2 is a diagram of an implementation of an example machine learning model.

FIG. 3 is a flowchart of a process for predicting patient post-operation improvement.

FIG. 4 is a block diagram of a computing device for predicting patient post-operation improvement.

### DETAILED DESCRIPTION

The present discourse is directed towards systems, methods, and computer program products that are configured to predict a level or degree of post-operation improvement for a patient. The present disclosure makes these prediction more accurately relative to conventional methods by training a machine learning model on particular sets of input data training data that to enable the machine learning model to configured to predict a level or degree of post-operation improvement for the patient. These particular inputs to the machine model include attributes reflecting both patients' perceptions and structural pathology. For example, patients' perception data, such as numerical values representing the patients' current feelings or pain level, and patients' morphological data, such as CT scans, are provided to machine learning models to predict how much the patients' conditions will improve after an operation. During training, the each set of training data can be labeled with a degree of patient post-operation improvement.

By virtue of the implementation of systems, methods, and computer program products described herein, techniques for patient post-operation improvement prediction using machine learning. Using data associated with both patients' perceptions and structural pathology increases the accuracy of the machine learning models. A large training sample ensures the machine learning models are resistant to the influence of outliers. Machine learning models generates reliable predictions in an efficient manner.

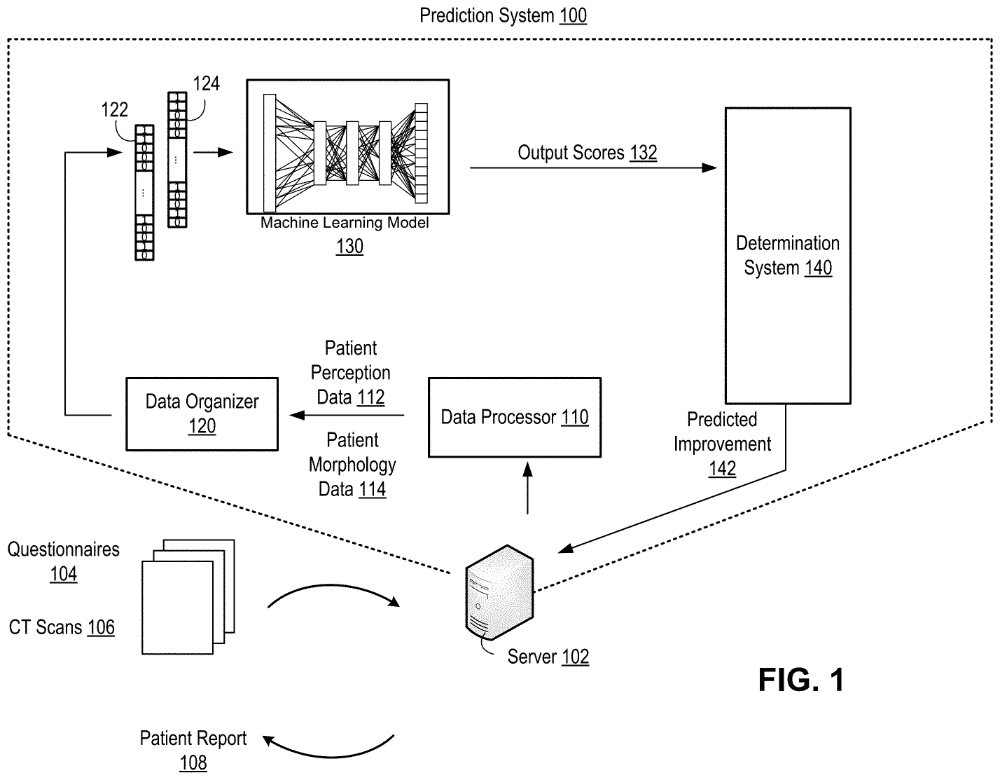

Referring now to FIG. 1, illustrated is an example prediction system 100. Generally, the prediction system generates a prediction about post-operation improvement for a patient based on both perception information and morphological information of a patient. In some embodiments, the prediction system 100 includes a server 102. The server 102 includes a data processor 110, a data organizer 120, a machine learning model 130, and a determination system 140. Each of the components of the prediction system 100 can be hosted on a single computer or hosted across multiple computers that are configured to communicate with each other using one or more networks.

In some embodiments, the prediction system 100 generates predictions associated with shoulder arthroplasty. In an example, the predictions generated are for patients with glenohumeral osteoarthritis (GHOA). In such an example, the predictions generated are associated with improvements in American Shoulder and Elbow Surgeons (ASES) scores.

The server 102 receives one or more questionnaires 104 and one or more computed tomography (CT) scans 106 as inputs and generates one or more patient reports 108 as outputs.

In some embodiments, the questionnaires 104 are converted into digital data. In such embodiments, the questionnaires 104 are represented as a table using standard data structures (e.g., an array, a list and/or the like). In some alternative embodiments, the questionnaires 104 includes answers keyed into the server 102 by either a patient or another human operator (e.g., a nurse, a physician and/or the like). In some other embodiments, the questionnaires 104 are images (e.g., scanned images of a set of paper questionnaires). Given the readily available Optical Character Recognition (OCR) algorithms, it is easy to convert a questionnaire 104 in an image format to a digitized table. In some embodiments, the one or more questionnaires 104 of a patient include information about the demographics and the patient's perception. In such embodiments, the questionnaires 104 include one or more of the following aspects of the patient: patient age at time of surgery, sex, operative side, preoperative Visual Analogue Scale (VAS) pain score, preoperative ASES responses to the ten activity-specific ASES function questions, and the ASES total score.

In some embodiments, the CT scans 106 are digital images (e.g., from a CT machine) with standard format (e.g., PNG, TIF, BMP and/or the like). In some embodiments, the one or more CT scans 106 of a patient include morphological information representative of the patient's structural pathology. In such embodiments, the CT scans 106 indicates one or more of the following preoperative morphological variables of the patient: Walch type, Goutallier classification, and the tangent sign.

The following discussion assumes the questionnaires 104 are digitized tables and the CT scans 106 are 2-dimensional (2D) digital images with standard format (e.g., PNG, TIF, BMP and/or the like).

In some embodiments, if the input questionnaires 104 and CT scans 106 are from more than one patients, each of the questionnaires 104 and/or the CT scans 106 contains information about the identity of the associated patient. In such embodiments, the information about the identity of the associated patient is a unique identifier, such as a unique patient ID. In an example, the unique patient ID is provided on the patient report. The unique patient ID distinguishes patients with the same name and ensures only the information related to the corresponding patient is used to generate the patient report 108.

In some embodiments, the patient report 108 of a patient includes identity information of the patient and information about predicted improvement in ASES scores for the patient.

In some embodiments, the questionnaires 104 and CT scans 106 received by the server 102 are provided as inputs to a data processor 110. In some embodiments, the data processor 110 extracts relevant data from the questionnaires 104 and CT scans 106, respectively, to generate the patient perception data 112 and the patient morphology data 114. In some embodiments, the data processor 110 assigns the unique patient ID as a header to the patient perception data 112 and the patient morphology data 114.

The data processor 110 generates patient perception data 112 based on extracted information from the questionnaires 104. In some embodiments, the data processor 110 parses information in the questionnaires 104 to generate the patient perception data 112. An example parsing process is found in the discussion below.

In some embodiments, the patient perception data 112 is a list. In an example, the questionnaire 104 is an ASES questionnaire used to calculate the total ASES score. For example, Question 14 of the ASES questionnaire reads “Is it difficult for you to lift 10 Ibs. (4.5 kg) above your shoulder?” while the answers are “Unable to do”, “Very difficult to do”, “Somewhat difficult” and “Not difficult”. In such an example, if the patient chooses “Somewhat difficult” as the answer, the element in the patient perception data 112 corresponding to Question 14 of the ASES questionnaire is a string value of “Somewhat difficult”.

The data processor 110 generates patient morphology data 114 based on extracted information from the CT scans 106. In some embodiments, the data processor 110 first normalizes the CT scans 106 to be 8-bit images. In some embodiments, the data processor 114 includes Mimics 14.1 to allow for reformatting of the CT scans 106 in the plane of the scapula. In such embodiments, glenoid morphology is determined based on the reformatted 2D axial CT scan images. In such embodiments, the patient morphology data 114 is the normalized and reformatted 2D CT scan images. In some other embodiments, the data processor 110 generates 3-dimensional (3D) CT reconstructions that analyze the scapula as a free body based on the 2D CT scan images. In such embodiments, the patient morphology data 114 is the 3D CT reconstructions. In an example, the patient morphology data 114 as the 3D CT reconstructions is a tensor (i.e. a 3D array).

In some embodiments, the data processor 110 presents, through an interface communicatively coupled with the server 102 (e.g., a display, a smartphone, a tablet device and/or the like), the CT scan images or the 3D reconstructions to one or more medical experts (e.g., surgeons) to obtain the morphological variables, such as Walch type, Goutallier classification, and the tangent sign. In some embodiments, the data processor 110 evaluated the CT scans for the morphological variables, such as Walch type, Goutallier classification, and the tangent sign based on a rule-based decision tree algorithm. In such embodiments, the rule-based decision tree algorithm is designed based on advice from one or more medical experts. In some embodiments, the patient morphology data 114 is a list including morphological variables such as the Walch type, Goutallier classification, and the tangent sign.

The server 102 provides the patient perception data 112 and the patient morphology data 114 to a data organizer 120 to generate, respectively, a first set of data indicative of one or more perceptions from a patient and a second set of data indicative of one or more morphological variables of the patient. In some embodiments, the first set of data is called the organized patient perception data 122 while the second set of data is called the organized patient morphology data 124.

In some embodiments, the data organizer 120 checks in the header the unique patient ID. In such embodiments, the data organizer 120 saves into a bundle implemented with some data structures (e.g. a nested list, a tuple and/or the like), for each unique patient ID, associated patient perception data 112 and associated patient morphology data 114. Each bundle represents data from a unique patient ID.

In some embodiments, the data organizer 120 generates the organized patient perception data 122 by converting non-numerical values in the patient perception data 112 in a bundle into numerical values. In an example, the patient perception data 112 is a list in which the element corresponding to Question 14 of the ASES questionnaire is one of “Unable to do”, “Very difficult to do”, “Somewhat difficult” and “Not difficult”. The options “Unable to do”, “Very difficult to do”, “Somewhat difficult” and “Not difficult” are converted into increasing numerical values. For example, “Unable to do” is converted to 0, “Very difficult to do” to 1.67, “Somewhat difficult” to 3.33 and “Not difficult” to 4. In such an example, the organized patient perception data 122 is a vector consisting of numerical values.

In some embodiments, where a bundle under a unique patient ID contains multiple pieces of patient perception data 112 (e.g., when a particular patient takes multiple questionnaires 104), the data organizer 120 generates a matrix as the organized patient perception data 122. In such embodiments, each row of the organized patient perception data 122 represents the numerical values corresponding to the answers to a questionnaire 104, and the rows of the matrix are ordered according to the importance and/or relevance of the corresponding questionnaires 104. In an example, the rows are rearranged by the data organizer 120 according to when the corresponding questionnaires 104 are taken by the particular patient. In such an example, the first row of the organized patient perception data 122 corresponds to answers to the most recent questionnaire 104. In some other embodiments, the data organizer 120 selects only the most recent piece of the patient perception data 112 and converts that into the organized patient perception data 122. In such embodiments, the organized patient perception data 122 is a vector consisting of numerical values.

In some embodiments, if the patient morphology data 114 is the normalized and reformatted 2D CT scans or the 3D reconstructions, the data organizer 120 generates the organized patient morphology data 124 by stacking (e.g., concatenating) the patient morphology data 114 in the same bundle to form a new tensor. The organized patient morphology data 124 is the tensor.

In some other embodiments, if the patient morphology data 114 is a list including morphological variables, the data organizer 120 generates the organized patient morphology data 124 by converting non-numerical values in the patient morphology data 114 in a bundle into numerical values. In some embodiments, each of the morphological variables (e.g., the Walch type, the Goutallier classification, and the tangent sign) is converted into one-hot encoding format. For example, Walch classification A1, A2, B1, B2, C are converted into [1, 0, 0, 0, 0], [0, 1, 0, 0, 0], [0, 0, 1, 0, 0], [0, 0, 0, 1, 0], and [0, 0, 0, 0, 1], respectively. In some embodiments, the one-hot encoded morphological variables are stacked, with appended 0's if necessary to make the encoded variables of the same length, by the data organizer 120 to form a matrix as the organized patient morphology data 124. The order of the stacking is consistent for all bundles. In some alternative embodiments, each morphological variable is converted into one numerical value and the organized patient morphology data 124 is a vector consisting of the numerical values.

The server 102 provides the organized patient perception data 122 and the organized patient morphology data 124 in the same bundle as input to a machine learning model 130 that has been trained to predict post-operation improvement of patient. The machine learning model 130 generates output scores 132 based on the input. In some embodiments, the output scores 132 represents likelihoods of which levels, categories, or classes the patient is predicted to be in. Example classes are Class A representing improvement in ASES Shoulder Assessment total score with 28 points and less, Class B representing improvement of 29-55 points, and Class C representing improvement of 45 points or more. In such embodiments, the machine learning model 130 is a classification model, such as a convolutional neural network (CNN), a logistic regression classifier, a random forest, a support vector machine (SVM), a hidden Markov model (HMM) and/or the like, or their combinations.

For example, when both the organized patient perception data 122 and the organized patient morphology data 124 are vectors, the organized patient perception data 122 and the organized patient morphology data 124 are conjoined (e.g., via concatenation) to form the input to a SVM. The SVM then predicts the improvement class (e.g., Class A, Class B or Class C) to which the patient belongs.

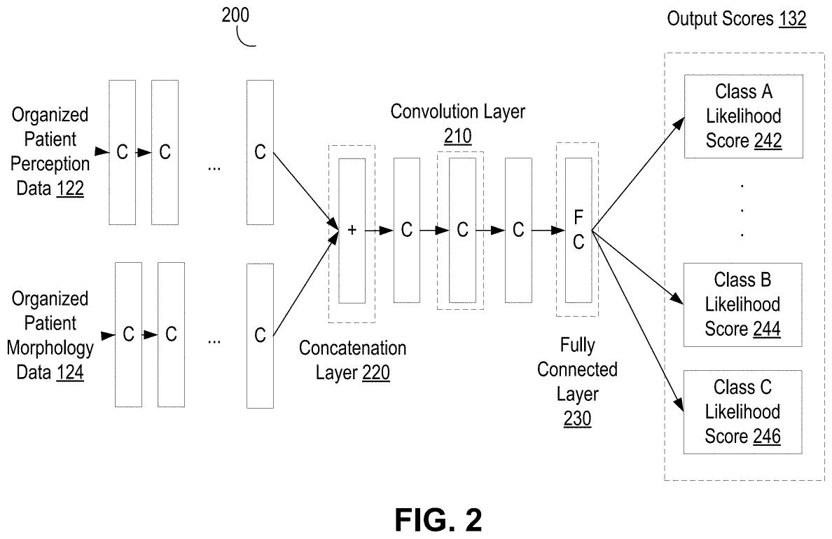

In some other embodiments, the output scores 132 represents the predicted improvements in ASES Shoulder Assessment total score. In such embodiments, the machine learning model 130 is a regression model, such as a linear regression model, an artificial neural network and/or the like, or their combinations. An example machine learning model 130 and detailed discussion about the output scores 132 are found below in FIG. 2.

The machine learning model 130 can be trained to predict the post-operation improvement of the patient in a number of different ways. For example, in some implementations, the machine learning model 130 can be trained by a training system on training data items that include patient perception data, patient morphology data, and a label indicating a known post-operation improvement level (or classification) for the patient from which the patient perception data and patient morphology data of the training data item was derived. The training system can process the training data item through the machine learning model 130 and then determine the difference between the output generated by the machine learning model 130 and the label of the training data item using a loss function. Then, the training system can update the parameters of machine learning model based on the difference between the output data generated by the machine learning model 130 and the labeled training data, which represents the ground truth. The training system can iteratively update the parameters of the machine learning model for each different training input of a library of training data input items until the loss function is optimized. In this manner, the machine learning model 130 can be trained to predict the post-operation improvement of a patient based on processing of input data that includes patient perception data and patient morphology data, each of which are described herein.

The server 102 provides output scores 132 as input to a determination system 140. The determination system 140 determines predicted improvement 142 based on the input and some criteria and/or threshold. In some embodiments, if the output score 132 is a single numerical value, the predicted improvement 142 is the output score 132 representing the improvement in ASES Shoulder Assessment total score. In some other embodiments, if the output scores 132 are a set of numerical values, the set of numerical values represent the likelihoods of improvement classes to which the patient belongs. In such embodiments, the determination system 140 determines predicted improvement 142 as the range indicated by the class of the patient, which corresponds to the class with the maximum likelihood score. The determination system 140 can also detect the unique patient ID and assigns the predicted improvement 142 accordingly.

The server 102 takes as input predicted improvement 142 and generates the patient report 108. In some embodiments, the patient report 108 includes recommendation to the patient and/or the physician whether to pursuit the operation or not, based on the predicted improvement 142. The patient report 108 is delivered to the patient and/or physician using some interface devices communicatively coupled with the server 102 (e.g., a display, a smartphone, a tablet device and/or the like). In some embodiments, the patient report 108 of a patient includes the unique patient ID of the patient and information about predicted improvement in the total ASES score for the patient. For example, a negative improvement in the total ASES score for the patient indicates that the patient's conditions may worsen after the operation and the recommendation is not to undergo the operation.

Referring now to FIG. 2, illustrated is a diagram of an implementation of an example machine learning model. More specifically, illustrated is a diagram of an implementation of an example convolutional neural network (CNN) 200. CNN 200 is a classification model used to predict to which improvement class the patient belongs. While CNN 200 includes certain features as described herein, these features are provided for the purpose of illustration and are not intended to limit the present disclosure.

CNN 200 has a dual head data reception structure where one reception head receives the organized patient perception data 122 while the other reception head receives the organized patient morphological data 124. In an example, as illustrated, the dual reception heads are successive convolution layers. The dual reception heads can have varying depth (i.e. number of convolution layers). In another example, each of the dual reception heads consists of multiple reception heads to receive different components of the input data. In some embodiments, if either the organized patient perception data 122 or the organized patient morphological data 124 is a vector, the input data in the vector form can skip the respective reception head and be provided as input directly to the concatenation layer 220.

The organized patient perception data 122 received by one of the reception head of CNN 200 includes the numerical representations of one or more of patient age at time of surgery, sex, operative side, preoperative Visual Analogue Scale (VAS) pain score, preoperative ASES responses to the ten activity-specific ASES function questions, and the ASES total score of the patient associated with the unique patient ID. In an example, the organized patient morphological data 124 received by one of the reception head of CNN 200 is the numerical representations of one or more of the morphological variables (e.g., Walch type, Goutallier classification, and the tangent sign) of the patient associated with the unique patient ID. In another example, the organized patient morphological data 124 received by one of the reception head of CNN 200 includes a tensor consisting of normalized and reformatted 2D CT scan images indicative of the morphological variables mentioned above of the patient associated with the unique patient ID. Additionally, or alternatively, the organized patient morphological data 124 received by one of the reception head of CNN 200 includes a tensor representing the 3D reconstructions using the 2D CT scans. In these examples, the values representing a 2D CT scan image can correspond to values representing a region of the image (sometimes referred to as a receptive field).

CNN 200 includes a plurality of convolution layers including the convolution layer 210. For example, CNN 200 performs an example convolution function based on CNN 200 providing some values as input to one or more neurons (not explicitly illustrated) included in the convolution layer 210. In some embodiments, each neuron is associated with a kernel (not explicitly illustrated). A kernel is representable as an array of values (e.g., a 4×5 matrix) that corresponds in size to the values provided as input to the neuron. In one example, a kernel may be configured to identify edges (e.g., horizontal lines, vertical lines, straight lines, and/or the like). In successive convolution layers, the kernels associated with neurons may be configured to identify successively more complex patterns (e.g., arcs, objects, and/or the like).

In some embodiments, CNN 200 performs a convolution function based on CNN 200 multiplying the values provided as input to each of the one or more neurons included in the convolution layer 210 with the values of the kernel that corresponds to each of the one or more neurons. For example, CNN 200 can multiply the values provided as input to each of the one or more neurons included in the convolution layer 210 with the values of the kernel that corresponds to each of the one or more neurons to generate a single value or an array of values as an output. In some embodiments, the collective output of the neurons of the convolution layer 210 is referred to as a convolved output. In some embodiments, where each neuron has the same kernel, the convolved output is referred to as a feature map.

In some embodiments, CNN 200 provides the outputs of each neuron of the convolution layer 210 to neurons of a downstream layer. For purposes of clarity, an upstream layer can be a layer that transmits data to a different layer (referred to as a downstream layer). For example, CNN 200 can provide the outputs of each neuron of the convolution layer 210 to corresponding neurons of a later convolution layer. In some embodiments, CNN 200 adds a bias value to the aggregates of all the values provided to each neuron of the downstream layer. In such an example, CNN 200 determines a final value to provide to each neuron of the downstream layer based on the aggregates of all the values provided to each neuron and an activation function (e.g., a sigmoid function, ReLU, leaky ReLU and/or the like) associated with each neuron of the downstream layer.

In some embodiments, a convolution layer has an appended max pooling layer (not explicitly illustrated). In some embodiments, the appended max pooling layer has a dimension (i.e., an amount of nodes) that is less than a dimension of an upstream layer (e.g. a preceding layer). The max pooling layer having a dimension that is less than a dimension of an upstream layer allows CNN 200 to consolidate the amount of data associated with the initial input and/or the output of an upstream layer to thereby decrease the amount of computations necessary for CNN 200 to perform downstream convolution operations. For example, CNN 200 performs a max pooling function based on CNN 200 providing the values output by the convolution layer 210 to corresponding neurons of the appended max pooling layer. In some embodiments, CNN 200 performs the max pooling function based on an aggregation function. In an example, CNN 200 performs the max pooling function by determining the maximum input among the values provided to a given neuron. In some embodiments, CNN 200 generates an output based on CNN 200 providing the values to each neuron of the max pooling layer, the output sometimes referred to as a pooled convolved output. In some alternatives embodiments, the max pooling layer is replaced by a min-pooling layer where CNN 200 performs the min-pooling function by determining the minimum input among the values provided to a given neuron, or by an average pooling layer function where CNN 200 performs the average pooling function by determining the average input (i.e. the mean input value) among the values provided to a given neuron.

CNN 200 includes at least one concatenation layer 220. The concatenation layer 220 conjoins two sets of input data generated from the dual reception heads by concatenating the two sets of data along the matching dimension. In an example, one reception head outputs a feature map of size (also referred to as shape) 4×2, while the other reception head outputs a pooled convolved output of size 7×2, the concatenation layer 220 takes the two sets of output and generates a concatenated data of size 12×2. The concatenated data is provided as input to a downstream layer. By the design of CNN 200, at least one of the dimension of the two sets of input data generated from the dual reception heads should match.

CNN 200 provides the output of each neuron of a preceding layer to fully connected layers 230. For example, CNN 200 provides the output of each neuron of a preceding layer to fully connected layers 230 to cause fully connected layers 230 to generate an output. In some embodiments, fully connected layers 230 are configured to generate an output associated with a prediction (sometimes referred to as a classification). The prediction includes an indication of the improvement class (e.g., Class A, Class B or Class C) that the associated patient belongs to. In some embodiments, the prediction is the output scores 132. The output scores 132 includes improvements class scores such as Class A likelihood score 242, Class B likelihood score 244 and Class C likelihood score 246. Each of the output scores 132 indicates the respective likelihood of the respective improvement class that the associated patient belongs to. In some embodiments, the output scores 132 is provided as input to the determination system 140 to determine the improvement class that the associated patient belongs to.

The CNN 200 can be modified into an artificial neural network to perform regression analysis on the input data. The output of the regression analysis model can be a single numerical value representative of the improvement in the total ASES score if the associated patient undergoes the operation. In an example, some of the convolution layers in the CNN 200 are replaced with linear layers, some of the max pooling layer in the CNN 200 are replaced with non-linearity layers and the fully connected layers 230 have an output of size one. A linear layer applies a linear transformation to the input data, and is capable of learning an offset and a rate of correlation. A non-linearity layer applies an activation function (discussed above) to the input data. The activation function is an element-wise operation. In such an example, the fully connected layers 230 generates an output score 132 consisting of only one numerical value representing the improvement in the total ASES score.

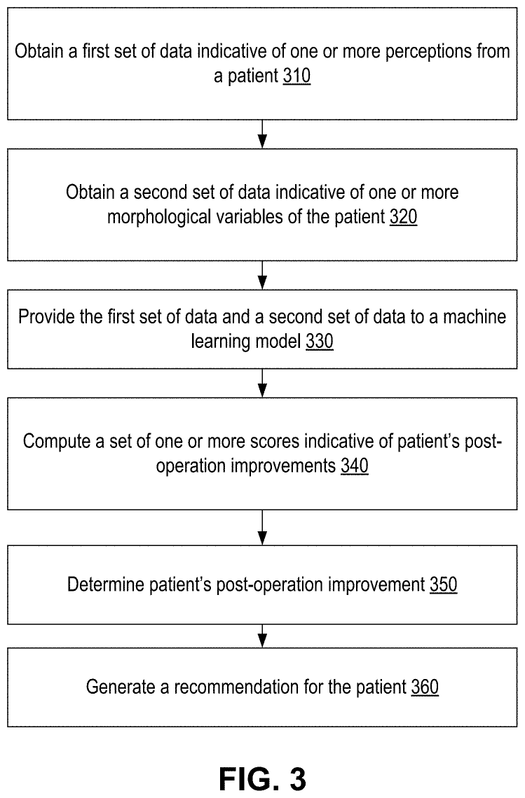

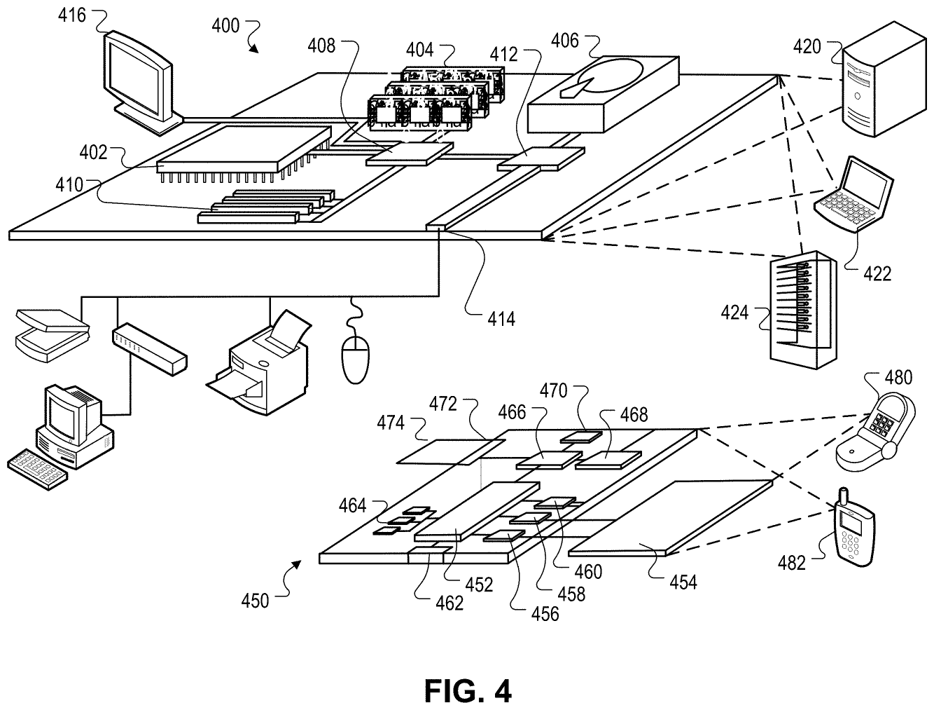

Referring now to FIG. 3, illustrated is a flowchart of a process 300 for predicting patient post-operation improvement using machine learning. In some embodiments, one or more of the steps described with respect to process 300 are performed (e.g., completely, partially, and/or the like) by the server 102. In some embodiments, the server 102 is the same as or similar to the server 420 described in FIG. 4. Additionally, or alternatively, in some embodiments one or more steps described with respect to process 300 are performed (e.g., completely, partially, and/or the like) by another device or group of devices separate from or including the server 102, such as the server 420.

With continued reference to FIG. 3, a first set of data indicative of one or more perceptions from a patient is obtained (block 310). In some embodiments, the first set of data indicative of one or more perceptions from the patient is the organized patient perception data 122 and the one or more perceptions include patient age at time of surgery, sex, operative side, preoperative VAS pain score, preoperative ASES responses to the ten activity-specific ASES function questions, or the ASES total score.

With continued reference to FIG. 3, a second set of data indicative of one or more morphological variables of the patient is obtained (block 320). In some embodiments, the second set of data indicative of one or more morphological variables of the patient is the organized patient morphology data 124 and the one or more morphological variables include Walch type, Goutallier classification, and the tangent sign.

With continued reference to FIG. 3, the first set of data and a second set of data to a machine learning model is provided to a machine learning model (block 330). In some embodiments, the machine learning model is the machine learning model 130. In some embodiments, the machine learning model is the CNN 200.

With continued reference to FIG. 3, a set of one or more scores indicative of patient's post-operation improvements is computed using the machine learning model (block 340). In some embodiments, the set of one or more scores is the output scores 132.

With continued reference to FIG. 3, the patient's post-operation improvement is determined (block 350). In some embodiments, the determining is performed by the determination system 140 based on the output scores 132.

With continued reference to FIG. 3, a recommendation for the patient is generated (block 360). In some embodiments, the recommendation is in the patient report 108 and the recommendation in the patient report 108 includes recommendation to the patient and/or the physician whether to pursuit the operation or not, based on determining the patient's post-operation improvement.

Referring now to FIG. 4, illustrated is a block diagram of a computing device 400 for predicting patient post-operation improvement using machine learning. Computing device 400 is intended to represent various forms of digital computers, such as laptops, desktops, workstations, personal digital assistants, servers, blade servers, mainframes, and other appropriate computers. Computing device 450 is intended to represent various forms of mobile devices, such as personal digital assistants, cellular telephones, smartphones, and other similar computing devices. Additionally, computing device 400 or 450 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 400 includes a processor 402, memory 404, a storage device 408, a high-speed interface 408 connecting to memory 404 and high-speed expansion ports 410, and a low speed interface 412 connecting to low speed bus 414 and storage device 408. Each of the components 402, 404, 408, 408, 410, and 412, are interconnected using various busses, and can be mounted on a common motherboard or in other manners as appropriate. The processor 402 can process instructions for execution within the computing device 400, including instructions stored in the memory 404 or on the storage device 408 to display graphical information for a GUI on an external input/output device, such as display 416 coupled to high speed interface 408. In other implementations, multiple processors and/or multiple buses can be used, as appropriate, along with multiple memories and types of memory. Also, multiple computing devices 400 can be connected, with each device providing portions of the necessary operations, e.g., as a server bank, a group of blade servers, or a multi-processor system.

The memory 404 stores information within the computing device 400. In one implementation, the memory 404 is a volatile memory unit or units. In another implementation, the memory 404 is a non-volatile memory unit or units. The memory 404 can also be another form of computer-readable medium, such as a magnetic or optical disk.

The storage device 408 is capable of providing mass storage for the computing device 400. In one implementation, the storage device 408 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 404, the storage device 408, or memory on processor 402.

The high speed controller 408 manages bandwidth-intensive operations for the computing device 400, while the low speed controller 412 manages lower bandwidth intensive operations. Such allocation of functions is exemplary only. In one implementation, the high-speed controller 408 is coupled to memory 404, display 416, e.g., through a graphics processor or accelerator, and to high-speed expansion ports 410, which can accept various expansion cards (not shown). In the implementation, low-speed controller 412 is coupled to storage device 408 and low-speed expansion port 414. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 400 can be implemented in a number of different forms, as shown in the figure. For example, it can be implemented as a standard server 420, or multiple times in a group of such servers. It can also be implemented as part of a rack server system 424. In addition, it can be implemented in a personal computer such as a laptop computer 422. Alternatively, components from computing device 400 can be combined with other components in a mobile device (not shown), such as device 450. Each of such devices can contain one or more of computing device 400, 450, and an entire system can be made up of multiple computing devices 400, 450 communicating with each other.

The computing device 400 can be implemented in a number of different forms, as shown in the figure. For example, it can be implemented as a standard server 420, or multiple times in a group of such servers. It can also be implemented as part of a rack server system 424. In addition, it can be implemented in a personal computer such as a laptop computer 422. Alternatively, components from computing device 400 can be combined with other components in a mobile device (not shown), such as device 450. Each of such devices can contain one or more of computing device 400, 450, and an entire system can be made up of multiple computing devices 400, 450 communicating with each other

Computing device 450 includes a processor 452, memory 464, and an input/output device such as a display 454, a communication interface 466, and a transceiver 468, among other components. The device 450 can also be provided with a storage device, such as a micro-drive or other device, to provide additional storage. Each of the components 450, 452, 464, 454, 466, and 468, are interconnected using various buses, and several of the components can be mounted on a common motherboard or in other manners as appropriate.

The processor 452 can execute instructions within the computing device 450, including instructions stored in the memory 464. The processor can be implemented as a chipset of chips that include separate and multiple analog and digital processors. Additionally, the processor can be implemented using any of a number of architectures. For example, the processor 410 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 450, such as control of user interfaces, applications run by device 450, and wireless communication by device 450.

Processor 452 can communicate with a user through control interface 458 and display interface 456 coupled to a display 454. The display 454 can be, for example, a TFT (Thin-Film-Transistor Liquid Crystal Display) display or an OLED (Organic Light Emitting Diode) display, or other appropriate display technology. The display interface 456 can comprise appropriate circuitry for driving the display 454 to present graphical and other information to a user. The control interface 458 can receive commands from a user and convert them for submission to the processor 452. In addition, an external interface 462 can be provide in communication with processor 452, so as to enable near area communication of device 450 with other devices. External interface 462 can provide, for example, for wired communication in some implementations, or for wireless communication in other implementations, and multiple interfaces can also be used.

The memory 464 stores information within the computing device 450. The memory 464 can be implemented as one or more of a computer-readable medium or media, a volatile memory unit or units, or a non-volatile memory unit or units. Expansion memory 474 can also be provided and connected to device 450 through expansion interface 472, which can include, for example, a SIMM (Single In Line Memory Module) card interface. Such expansion memory 474 can provide extra storage space for device 450, or can also store applications or other information for device 450. Specifically, expansion memory 474 can include instructions to carry out or supplement the processes described above, and can include secure information also. Thus, for example, expansion memory 474 can be provide as a security module for device 450, and can be programmed with instructions that permit secure use of device 450. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 464, expansion memory 474, or memory on processor 452 that can be received, for example, over transceiver 468 or external interface 462.

Device 450 can communicate wirelessly through communication interface 466, which can include digital signal processing circuitry where necessary. Communication interface 466 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 468. In addition, short-range communication can occur, such as using a Bluetooth, Wi-Fi, or other such transceiver (not shown). In addition, GPS (Global Positioning System) receiver module 470 can provide additional navigation- and location-related wireless data to device 450, which can be used as appropriate by applications running on device 450.

Device 450 can also communicate audibly using audio codec 460, which can receive spoken information from a user and convert it to usable digital information. Audio codec 460 can likewise generate audible sound for a user, such as through a speaker, e.g., in a handset of device 450. Such sound can include sound from voice telephone calls, can include recorded sound, e.g., voice messages, music files, etc. and can also include sound generated by applications operating on device 450.

The computing device 450 can be implemented in a number of different forms, as shown in the figure. For example, it can be implemented as a cellular telephone 480. It can also be implemented as part of a smartphone 482, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

In the foregoing description, aspects and embodiments of the present disclosure have been described with reference to numerous specific details that can vary from implementation to implementation. Accordingly, the description and drawings are to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising,” in the foregoing description or following claims, what follows this phrase can be an additional step or entity, or a sub-step/sub-entity of a previously-recited step or entity.

In this detailed description, numerous specific details are set forth in order to provide a thorough understanding of the various described embodiments. However, it will be apparent to one of ordinary skill in the art that the various described embodiments can be practiced without these specific details. In other instances, well-known methods, procedures, components, circuits, and networks have not been described in detail so as not to unnecessarily obscure aspects of the embodiments.

In preceding detailed description, numerous specific details are set forth in order to provide a thorough understanding of the present disclosure for the purposes of explanation. It will be apparent, however, that the embodiments described by the present disclosure can be practiced without these specific details. In some instances, well-known structures and devices are illustrated in block diagram form in order to avoid unnecessarily obscuring aspects of the present disclosure.

Specific arrangements or orderings of schematic elements, such as those representing systems, devices, modules, instruction blocks, data elements, and/or the like are illustrated in the drawings for ease of description. However, it will be understood by those skilled in the art that the specific ordering or arrangement of the schematic elements in the drawings is not meant to imply that a particular order or sequence of processing, or separation of processes, is required unless explicitly described as such. Further, the inclusion of a schematic element in a drawing is not meant to imply that such element is required in all embodiments or that the features represented by such element may not be included in or combined with other elements in some embodiments unless explicitly described as such.

Further, where connecting elements such as solid or dashed lines or arrows are used in the drawings to illustrate a connection, relationship, or association between or among two or more other schematic elements, the absence of any such connecting elements is not meant to imply that no connection, relationship, or association can exist. In other words, some connections, relationships, or associations between elements are not illustrated in the drawings so as not to obscure the disclosure. In addition, for ease of illustration, a single connecting element can be used to represent multiple connections, relationships or associations between elements. For example, where a connecting element represents communication of signals, data, or instructions (e.g., “software instructions”), it should be understood by those skilled in the art that such element can represent one or multiple signal paths (e.g., a bus), as may be needed, to affect the communication.

Although the terms first, second, third, and/or the like are used to describe various elements, these elements should not be limited by these terms. The terms first, second, third, and/or the like are used only to distinguish one element from another. For example, a first contact could be termed a second contact and, similarly, a second contact could be termed a first contact without departing from the scope of the described embodiments. The first contact and the second contact are both contacts, but they are not the same contact.

The terminology used in the description of the various described embodiments herein is included for the purpose of describing particular embodiments only and is not intended to be limiting. As used in the description of the various described embodiments and the appended claims, the singular forms “a,” “an” and “the” are intended to include the plural forms as well and can be used interchangeably with “one or more” or “at least one,” unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this description specify the presence of stated features, integers, steps, operations, elements, and/or components, but do not preclude the presence or addition of one or more other features, integers, steps, operations, elements, components, and/or groups thereof.

As used herein, the terms “communication” and “communicate” refer to at least one of the reception, receipt, transmission, transfer, provision, and/or the like of information (or information represented by, for example, data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and transmits the processed information to the second unit. In some embodiments, a message may refer to a network packet (e.g., a data packet and/or the like) that includes data.

As used herein, the term “if” is, optionally, construed to mean “when”, “upon”, “in response to determining,” “in response to detecting,” and/or the like, depending on the context. Similarly, the phrase “if it is determined” or “if [a stated condition or event] is detected” is, optionally, construed to mean “upon determining,” “in response to determining,” “upon detecting [the stated condition or event],” “in response to detecting [the stated condition or event],” and/or the like, depending on the context. Also, as used herein, the terms “has”, “have”, “having”, or the like are intended to be open-ended terms. Further, the phrase “based on” is intended to mean “based at least partially on” unless explicitly stated otherwise.

Some embodiments of the present disclosure are described herein in connection with a threshold. As described herein, satisfying a threshold can refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

What is claimed is:

1. A method, comprising:
obtaining, by one or more computers, a first set of data indicative of one or more perceptions of a patient;
obtaining, by one or more computers, a second set of data indicative of one or more morphological variables of the patient;
generating, by one or more computers, input data that comprises the first data set and the second data set;
providing, by one or more computers, the generated input data to a machine learning model that has been trained to generate output data indicative of a patient's post-operation improvement based on processing of input data that includes (i) a first data set indicative of one or more perceptions of a patient and (ii) second data indicative of one or more morphological variables of the patient;
processing, by one or more computers, the provided input data through the machine learning model in order to generate output data that includes a set of one or more scores indicative of patient's post-operation improvements based on the provided first set of data and the second set of data;
determining, by one or more computers and based on the generated output data, a level of post-operation improvement for the patient; and
generating, by one or more computers, data corresponding to a recommendation for the patient whether to undergo the operation or not, based on the determined level of poser operation improvement for the patient.

2. The method of claim 1, wherein the first set of data includes numerical representations of a patient age at time of surgery, sex, operative side, preoperative VAS pain score, preoperative ASES responses to the ten activity-specific ASES function questions, or an ASES total score.

3. The method of claim 1, wherein the second set of data can includes a 2D CT scan that indicates a Walch type, a Goutallier classification, or a tangent sign.

4. The method of claim 1, wherein the second set of data can includes a 3D CT reconstruction that indicates one or more of a Walch type, a Goutallier classification, or a tangent sign.

5. The method of claim 1, wherein the second set of data includes a numerical representation of a Walch type, a Goutallier classification, or a tangent sign.

6. The method of claim 1, wherein the machine learning model is one of or a combination of a support vector machine (SVM), a random forest, a logistic regression classifier, a hidden Markov model, a linear regression model.

7. The method of claim 1, wherein the machine learning model is a convolutional neural network; and
wherein processing, by one or more computers, the provided input data through each layer of the machine learning model comprises:
processing, by one or more computers, the provided input data through each layer of the machine learning model the generated input data through each layer of the convolutional neural network.

8. A system, comprising:
at least one processor, and
at least one non-transitory storage media storing instructions that, when executed by the at least one processor, cause the at least one processor to perform operations, the operations comprising:
obtaining, by one or more computers, a first set of data indicative of one or more perceptions of a patient;
obtaining, by one or more computers, a second set of data indicative of one or more morphological variables of the patient;
generating, by one or more computers, input data that comprises the first data set and the second data set;
providing, by one or more computers, the generated input data to a machine learning model that has been trained to generate output data indicative of a patient's post-operation improvement based on processing of input data that includes (i) a first data set indicative of one or more perceptions of a patient and (ii) second data indicative of one or more morphological variables of the patient;
processing, by one or more computers, the provided input data through the machine learning model in order to generate output data that includes a set of one or more scores indicative of patient's post-operation improvements based on the provided first set of data and the second set of data;
determining, by one or more computers and based on the generated output data, a level of post-operation improvement for the patient; and
generating, by one or more computers, data corresponding to a recommendation for the patient whether to undergo the operation or not, based on the determined level of poser operation improvement for the patient.

9. The system of claim 8, wherein the first set of data includes a numerical representation a patient age at time of surgery, sex, operative side, preoperative VAS pain score, preoperative ASES responses to the ten activity-specific ASES function questions, or an ASES total score.

10. The system of claim 8, wherein the second set of data includes a 2D CT scan that indicates a Walch type, a Goutallier classification, or a tangent sign.

11. The system of claim 8, wherein the second set of data includes a 3D CT reconstruction that indicates a Walch type, a Goutallier classification, or a tangent sign.

12. The system of claim 8, wherein the second set of data includes a numerical representation a Walch type, a Goutallier classification, or a tangent sign.

13. The system of claim 8, wherein the machine learning model is one of or a combination of a support vector machine (SVM), a random forest, a logistic regression classifier, a hidden Markov model, a linear regression model.

14. The system of claim 8, wherein the machine learning model is a convolutional neural network; and
wherein processing, by one or more computers, the provided input data through each layer of the machine learning model comprises:
processing, by one or more computers, the provided input data through each layer of the machine learning model the generated input data through each layer of the convolutional neural network.

15. A non-transitory computer-readable storage medium comprising at least one program for execution by at least one processor of a first device, the at least one program including instructions which, when executed by the at least one processor, cause the first device to perform operations, the operations comprising:
obtaining a first set of data indicative of one or more perceptions of a patient;
obtaining a second set of data indicative of one or more morphological variables of the patient;
generating the input data that comprises the first data set and the second data set;
providing the generated input data to a machine learning model that has been trained to generate output data indicative of a patient's post-operation improvement based on processing of input data that includes (i) a first data set indicative of one or more perceptions of a patient and (ii) second data indicative of one or more morphological variables of the patient;
processing the provided input data through the machine learning model in order to generate output data that includes a set of one or more scores indicative of patient's post-operation improvements based on the provided first set of data and the second set of data;
determining, based on the generated output data, a level of post-operation improvement for the patient; and
generating data corresponding to a recommendation for the patient whether to undergo the operation or not, based on the determined level of poser operation improvement for the patient.

16. The computer-readable medium of claim 15, wherein the first set of data includes a numerical representation of a patient age at time of surgery, sex, operative side, preoperative VAS pain score, preoperative ASES responses to the ten activity-specific ASES function questions, or an ASES total score.

17. The computer-readable medium of claim 15, wherein the second set of data includes a 2D CT scan that indicates a Walch type, a Goutallier classification, or a tangent sign.

18. The computer-readable medium of claim 15, wherein the second set of data includes 3D CT reconstruction that indicates a Walch type, a Goutallier classification, or a tangent sign.

19. The computer-readable medium of claim 15, wherein the second set of data includes a numerical representation of a Walch type, a Goutallier classification, or a tangent sign.

20. The computer-readable medium of claim 15, wherein the machine learning model is a convolutional neural network; and
wherein processing, by one or more computers, the provided input data through each layer of the machine learning model comprises:
processing, by one or more computers, the provided input data through each layer of the machine learning model the generated input data through each layer of the convolutional neural network.
